(19) United States
(12) Patent Application Publication
(10) Pub. No.: US 2005/0154251 A1
(43) Pub. Date: Jul. 14, 2005

(54) Method and apparatus for correction of functional state of person

(75) Inventors: Alexander V. Martynenko, Kharkiv (UA); Kevin L. Ockunzzi, Brooklyn, OH (US); Mykola I. Yabluchanskiy, Kharkiv (UA); Vyacheslav I. Shulgin, Kharkiv (UA)

Correspondence Address: Christopher B. Fagan, Esq.;Fay, Sharpe, Fagan, Minnich & McKee, LLP, 1100 Superior Avenue, Seventh Floor, Cleveland, OH, 44114-2518, US

(73) Assignee: Micromedica Technologies LLC.

(21) Appl. No.: 11/031,782

(22) Filed: Jan. 7, 2005

(30) Foreign Application Priority Data
Jan. 12, 2004 (RU) 2004100969
Jan. 8, 2004 (UA) 2004010059

(51) Int. Cl. A61M 21/00; A61B 5/04

(52) U.S. Cl. 600/27; 128/905

(57) ABSTRACT

A method and apparatus for correction of a functional state of a subject organism is provided. In one embodiment, the method includes: a) measuring one or more biosignals reflecting a current functional state of the subject organism, b) transforming and processing the one or more measured biosignals to provide a current generalized index of health of the subject organism, c) evaluating a deviation of the current generalized index of health from a predetermined predicted optimal generalized index of health, d) forming of external effect on the subject organism using a controllable factor of effect having one or more controllable parameters, and e) regularly repeating the external effect until the deviation of the current generalized index of health from the predicted optimal generalized index of health is minimized and stabilized. The subject organism may be a patient. The patient may be a person.

This application claims the benefit of Ukrainian Patent Application Ser. No. 2004010059, filed on Jan. 8, 2004, and Russian Patent Application Ser. No. 2004100969, filed on Jan. 12, 2004. The disclosure of both of these patent applications is incorporated herein by reference.

### BACKGROUND

The present exemplary embodiments relate to medicine and medical techniques. They find particular application in conjunction with devices and methods of control and can be used for bioadaptive correction of functional state of a person, and will be described with particular reference thereto. However, it is to be appreciated that the present exemplary embodiments are also amenable to other like applications.

By way of background, it is well known that humans have natural resources of strength in different systems and that these resources allow the functional state of a person to change. This allows an organism to function even in very difficult conditions. Functional data is the basis for qualitative conclusion about a state of a patient that gives information about a choice of treatment strategy and how a patient should act. According to experience, effectiveness of treatment can be higher if the individuality of a patient is considered. The patient's individuality can be revealed with the help of different exercise testing (see Yabluchansky N., et al., Interpretation of clinical physiology of heart, Kharkov, 2001, 168 pages (in Russian)). It is preferable that treatment results in an optimal state of a patient, rather than some standard state. The optimal state can be reached with help of common efforts of a patient and a doctor.

For example, the functional state of a person can be corrected by sound with optimization of parameters of external influence on an organism (see Soviet Union patent document no. SU 1780716, 15 Dec. 1992). This may include recording biopotentials of physiological parameters, transformation and processing of received information, and calculation of characteristic parameters of the biosignal that is transformed into a drive signal and signals of external influence. External influence, for example, sound is being chosen from previously recorded phonograms that are distinguished by rhythm and key.

However, according to experience, prerecorded phonograms are random and may not completely correspond to individual peculiarities of an organism. This decreases the effectiveness of psychophysical correction of a person using the external influence of physical factors, such as sound.

Recording of biopotentials and formation of sound influence depending on biosignals is also a familiar method of influence on an organism. The sound influence is polyphonic (see Russian Federation patent document no. RU 2 143 839, 10 Jan. 2000). That is, every sound is determined by a corresponding biosignal where the quantity of recorded biopotentials determines the quantity of musical instruments for playing corresponding parts. This method provides an effect on an organism and simultaneously registers several biosignals and their correlation.

But this method is primarily for passive recording of sound influence on biosignals and can be used for receiving information that will be in the form of recommendations. This substantially decreases the effectiveness of this method and limits its functional possibilities.

Another method of influence on an organism includes recording of biopotentials of physiological parameters, transformation and processing of received information, calculation of typical generalized parameter of biosignal which is being transformed into a driving signal, and forming an external sound influence (see Russian Federation patent document no. RU 2 096 990, 27 Nov. 1997). The external sound influence is performed by generating musical sounds by means of parametric change of pitch, loudness, and length. This type of external sound influence (i.e., musical therapy) has been used for treatment of sleep disturbances of more than 200 patients.

But as in the previous case, in this method the patient acts passively in conditions of external influence. This substantially decreases the effectiveness of psychophysical correction of physiological state and restricts functional resources of the method. Another disadvantage of this method, as well as the other methods, is that there is only one kind of external influence applied. Namely, only sound is applied as an external influence.

A telemetric device for control and diagnostics of the functional state of a person is also well known (see Russian Federation patent document no. RU 2 175 212, 27 Oct. 2001). This device includes a transmitting component that is placed on a body of a person, blocks of recording of physiological signals and electrodes for recording of biopotentials, a block of prior amplification, a block of coding and infrared light, a block of decoding of signals, and a block of formation of outgoing informational signals. The coding and decoding blocks contain pulse-time modulator and pulse-time demodulator correspondingly and each contains a scheme of logic control and synchronization with quartz generator for stabilization of base frequency. Such construction of the device allows enlargement of its functional resources, provides control and diagnostics according to several simultaneously controlled physiological indexes taken from a general set of indexes, and decreases time of data reception of diagnostics of different kinds of physiological states.

However, the above mentioned device is complex and includes complicated equipment. Normal operations depend on a number of factors. The operating regime of the device is determined by taking into account optimization of the correlation between the power of infrared light, required reach, sensitivity of image devices, protection rate from hindrances, and parameters of a self-contained power supply associated with the transmitting part of the device. Additionally, as mentioned above, the functional potential of the device is limited by control and diagnostics.

A computer device for medical diagnostics that is also well known includes a high frequency generator with electrodes for modified tetrapolar rheography of two channels (see Russian Federation patent document no. RU 2 145 792, 27 Feb. 2000). Each channel includes a differential amplifier, a linear detector of half-period value, an amplifier of impedance plethysmogram, three filters of lower frequency, and two analogous differential devices. Mechanisms of summation and subtraction are turned on between the channels. Additionally, the device contains a channel of phonocardiogram. Outputs of the phonocardiogram are connected up with inputs of a many channeled analogous digital transformer within a computer. The application of this computer device (i.e., polycardiograph) allows more thorough examinations of patients, provides an algorithm using computer resources, and provides a printed conclusion about the state of a patient.

However, like in previous cases, diagnostics of the state of a patient is carried out when the patient stays calm and one functional test is carried out, such as isometric leg exercises. This decreases the effectiveness of diagnostics substantially and limits functional resources of the device.

Another device for correction of a functional state of a person includes sensors of biosignals, an amplifier, a block of prior processing of signals with outputs connected to an input of a switchboard, a block of recording, a block of a control board for measuring biosignal parameters, a block of control of an environment and a consistently connected and controlled sound generator with an input connected to an input of a block of signals synthesizing biological feedback (see Soviet Union patent document no. 1742204, 7 Jul. 1992). The device also includes a mixer, an amplifier, an acoustic transmitter, and a block of illumination where signals of biological feedback are displayed on a display. Synchronization and operation of the blocks are provided by a control board through tires of synchronization and connection. Parameters of the environment (e.g., sound, color, and brightness) help to implement correlation of the functional state of a person and are automatically selected in the process of operation of the device. The device is used to apply the method when a patient is observing an averaged meaning of a chosen parameter and its deviation from a predicted parameter and trying to decrease deviations of an averaged meaning of a parameter from a predicted meaning by means of self-regulation of his state. The device may provide increased effectiveness of correction of functional state by means of optimization of parameters of the environment in the process of correction.

However, the above mentioned device operates with only one biosignal, such as electric brain and heart activity, temperature, electric resistance of skin, change of chest volume during breathing, change of optical density of tissues, etc. This limits the functional resources of this method.

The present exemplary embodiment contemplates a new and improved method and apparatus that resolves the above-referenced difficulties and others. For example, the exemplary embodiment may increase the use of psychophysical resources of a person for correction of the person's functional state, may simplify the device, and/or may increase its functional potential.

### BRIEF DESCRIPTION

In one aspect, a method of correction of a functional state of a subject organism is provided. In one embodiment, the method includes: a) measuring one or more biosignals reflecting a current functional state of the subject organism, b) transforming and processing the one or more measured biosignals to provide a current generalized index of health of the subject organism, c) evaluating a deviation of the current generalized index of health from a predetermined predicted optimal generalized index of health, d) forming of external effect on the subject organism using a controllable factor of effect having one or more controllable parameters, and e) regularly repeating the external effect until the deviation of the current generalized index of health from the predicted optimal generalized index of health is minimized and stabilized.

In another aspect, an apparatus for correction of a functional state of an organism is provided. In one embodiment, the apparatus includes: a sensor adapted to measure a biosignal reflecting a current functional state of the organism, an amplifier in communication with the sensor to intensify the biosignal, a bandpass filter in communication with the biosignal amplifier to isolate a portion of the biosignal associated with a predetermined frequency range, an electronic switchboard in communication with the bandpass filter for switching the biosignal, a microprocessor in communication with the electronic switchboard to receive the switched biosignal from the electronic switchboard and to provide feedback to control operation of the electronic switchboard, an optical distributor in communication with the microprocessor, an interface block in communication with the optical distributor, a computer in communication with the interface block, and internal software located within at least one of the microprocessor and the computer that provides measuring, adaptation, and automatic control of one or more parameters of the biosignal.

In still another aspect, a method of correction of a physiological state of a subject organism is provided. In one embodiment, the method includes: a) providing a predicted optimal generalized index of health for the subject organism, b) recording a biosignal reflecting a current physiological state of the subject organism, c) transforming and processing the measured biosignal to determine a current generalized index of health of the subject organism, d) evaluating a difference between the current generalized index of health and the predicted optimal generalized index of health, and e) forming of external influence on the subject organism while observing the current generalized index of health by choosing a controllable factor of effect and values for controllable parameters associated with the corresponding controllable factor that change the current generalized index of health in a direction of the predicted optimal generalized index of health.

Further scope of the applicability of the present exemplary embodiments will become apparent from the detailed description provided below. It should be understood, however, that the detailed description and specific examples, while indicating preferred embodiments of the invention, are given by way of illustration only, since various changes and modifications within the spirit and scope of the invention will become apparent to those skilled in the art.

### BRIEF DESCRIPTION OF THE DRAWINGS

The present exemplary embodiments exist in the construction, arrangement, and combination of the various parts of the apparatus, and steps of the method, whereby the objects contemplated are attained as hereinafter more fully set forth, specifically pointed out in the claims, and illustrated in the accompanying drawings in which:

FIG. 1 is a fundamental scheme of an exemplary embodiment of a method for correction of a functional state of a person;

FIG. 2 is a fundamental scheme of an exemplary embodiment of an apparatus for correction of a functional state of a person; and

FIGS. 3A and 3B are a block diagram of an exemplary embodiment of operations and software support for an apparatus for correction of a functional state of a person.

### DETAILED DESCRIPTION

One aspect of an exemplary embodiment improves a method of an effect on an organism, such as a person. Presuming the existence of a difficult psychophysical state of a person, the change of biosignals, such as blood pressure, cardiac output, temperature, etc., is provided as a result of predetermination of a predicted optimal generalized index of health for the person, evaluation of deviation of a current generalized index of health of the person from the predicted optimal generalized index, exposing the person to an effect while checking the state of the person by choosing a factor and/or a parameter that causes the current generalized index of health of the person change in a direction of the predicted optimal generalized index, evaluation of a difference between the predicted optimal generalized index and the current generalized index, visualization of the difference between the predicted optimal generalized index and the current generalized index, for example, on a screen of a monitor, and regular repeats of the effect until minimization and stabilization of the difference between the predicted optimal generalized index and the current generalized index. Due to this optimization, an approximation to an optimal functional state of a definite patient is possible.

Another aspect of an exemplary embodiment improves an apparatus for correction of a functional state of a person. The use of an integral generalized index of health of a person and improved use of psychophysical resources of a person in order to influence a physiological state of the person is provided as a result of execution of a block of recording, numbering, and measuring of parameters of biosignals and adaptation and automatic control in the form of a microprocessor with internal software. A first output of the microprocessor is in communication with an input of a digital switchboard to provide feedback to control the digital switchboard. A second output of the microprocessor is in communication with an input of a block of optical distributor. An output of the block of optical distributor is connected to an input of an interface block and a computer. An output of the computer is connected to an input of a PC. A software support of the computer provides transmission, preservation, and further processing of a received biosignal and is aimed at its visualization, calculation of its parameters and indexes in order to calculate variability, spectrum formation, and determination of indexes of variability in the spectrum area with the possibility of processing of a biosignal in actual time for calculation of actual state of balance of regulatory links in an organism of a person. Due to this effective correction of functional parameters, a patient approaching optimal meanings typical for a definite person may be carried out in actual time.

Yet another aspect of an exemplary embodiment provides a method of correction of a functional state of a person. The method includes influencing external factors on an organism, recording a biosignal of a physiological state of the organism, transformation and processing of received information with calculation of a typical generalized parameter of the biosignal, and formation of external influence. First, a predicted optimal generalized index of health for a definite person is necessary to determine and evaluate deviation of a current index of health of a person from the predicted optimal generalized index. Then, an effect (i.e., external factors) is applied to a person while observing the person's state by choosing external factors and parameters that change the current index of health (i.e., state) of a person change in direction of predicted optimal generalized index. The difference between the predicted optimal generalized index and the current index of health is evaluated and visualized, for example, on a screen of a monitor. The effect is repeated periodically until the difference between the predicted optimal generalized index and the current index of health is minimized and stabilized. Some examples of the external factors of effect used include music, light, temperature, exercises, and modulated breathing. The periodic effect may be completed, for example, in the process of 5 to 20 sessions. The duration of each session may be five to fifteen minutes. The quantity and duration of sessions are determined while taking into account the current state of the person and goals of correction.

Still another aspect of an exemplary embodiment provides an apparatus for correction of a functional state of a person. In this embodiment, the apparatus includes sensors of biosignals, amplifiers of biosignals, blocks of preprocessing of signals outputs of which are in communication with an input of a switchboard, a block of recording, a block of numbering of biosignal, a block of measuring of parameters of biosignals, blocks of adaptation, and self-tuning. The blocks of recording, numbering, measuring of parameters of biosignals, adaptation and self-tuning are provided in the form of a microprocessor with an inner software support. An input of the microprocessor is in communication with an output of a digital switchboard. An output of the microprocessor is also in communication with the digital switchboard to provide feedback. Another output of the microprocessor is in communication with an input of a block of optical distributor. An output of the block of optical distributor is in communication with an input of an interface block and a computer. An output of the computer is communication with an input of a PC. A software support of the computer provides transmission, preservation and further processing of the received biosignal and is aimed at its visualization, calculation of its parameters and indexes in order to calculate variability, spectrum formation and determination of indexes of variability in the spectrum area with a possibility of processing of biosignal in actual time for calculation of actual state of balance of regulatory links in an organism of a person.

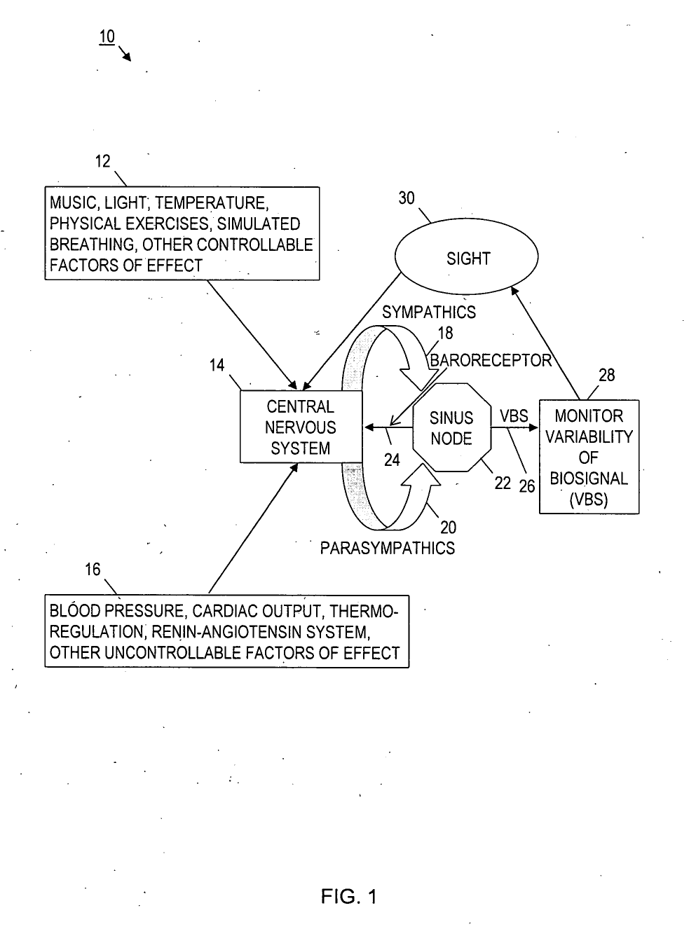

Referring now to the drawings, wherein the showings are for purposes of illustrating the preferred embodiments of the invention only and not for purposes of limiting same. FIG. 1 provides a functional scheme of an exemplary embodiment of a method 10 for correction of a functional state of a person includes controllable factors of effect 12, a central nervous system block 14, uncontrollable factors of effect 16, sympathetic regulation 18, parasympathetic regulation 20, a sinus node 22, baroreceptors 24, a variability of biosignal or biological signal (VBS) 26, a monitor VBS block 28, and a sight block 30.

The uncontrollable factors of effect 12 are homeostatic, internal, and physiological factors that define reactions of the central nervous system 14. The sympathetic regulation 18 is a vegetative nervous system that provides a sympathetic link between the central nervous system 14 and the sinus node 22. The parasympathetic regulation 20 is a vegetative nervous system that provides a parasympathetic link between the central nervous system 14 and the sinus node 22. The baroreceptors 24 represent baroreceptors of arterial vessels and provide feedback from the sinus node 22 to the central nervous system 14. The VBS 26 is a signal representing the variability of heart rate, pulse, etc. The monitor VBS block 28 is a device that reflects the VBS on a screen (e.g., visualization on a monitor). The sight block 30 represents a patient's visual perception of the VBS on the monitor.

The method 10 includes integral evaluation of a functional state of an organism, such as a person (i.e., patient), where links of regulation of the person's autonomous nervous system and an effect of the person's central nervous system 14 are involved in the formation of a registered and analyzed biosignal. At the same time, an optimizing effect on the person's health takes place due to biological feedback that is carried out through a conscious effect of the central nervous system 14 on a sinus node 22, taking into account factors of the environment that influence the health state of a person.

At the same time, the patient is observing an averaged meaning of a chosen parameter and its deviation from a predicted parameter and is trying to decrease deviations between the averaged meaning and the predicted meaning of the parameter by self-regulation of his/her state.

The method considers an optimal generalized index of health of a definite person as a typical generalized parameter and suggests that the person is affected by a definite factor 12 that is chosen in advance from a number of factors, such as music, light, temperature, exercises, modulated or simulated breathing, etc. Parameters of such effect are determined in advance.

The apparatus includes main blocks that are made in the form of a microprocessor and an optional mini PC. The safety of the patient from electricity is provided by means of an optical distributor. The program part of the apparatus provides broad functional possibilities because it allows use of different sensors of the biosignal, allows optimal tuning in order to receive a signal of the best quality, and implements control of biosignals and adaptation of the apparatus to changes in parameters of a signal.

The apparatus is an industrial device because it includes modern equipment using the latest technologies and technical means. The apparatus can be used for correction of the functional state of patients in medical institutions in order to reach or approach the optimal state and also for optimization of the functional state of people that are performing in competitive sports or similar activities.

Exemplary applications of the method and apparatus are described in the following examples.

### EXAMPLE 1

A male patient (K) was 56 years old and diagnosed with coronary heart disease, stable exertional angina, atherosclerosis, and full left anterior block of bundle. He had general weakness and rapid fatigability.

The patient was linked up with the apparatus for correction of functional state by four electrodes placed on his arms and legs. The cycles of inhalation-exhalation per minute (i.e., frequency of breathing) were calculated imperceptibly for the patient during the recording of biopotentials. The frequency of cycles of inhalation-exhalation was sixteen times per minute. During the recording of an electrocardiogram signal, the expected optimal and the actual position of the generalized index of health of the patient was observed on a monitor of the apparatus.

After switching on of the metronome with frequency of sixteen signals per minute, the patient was asked to breath in accordance with the signals of the metronome. The generalized index of health deviated in the direction from the optimal position during the breathing according to the metronome. When the frequency of the metronome and the coordinated breathing changed from thirteen cycles per minute to nineteen cycles per minute, it was noted that the expected index was moving towards the actual generalized index when the frequency of the coordinated with the metronome breathing was nineteen cycles per minute. The patient was asked to breath with a frequency of nineteen cycles per minute for ten minutes. As a result, the distance between the expected and the actual position of the generalized index of health was observed on the monitor to decrease by half.

Ten sessions of treatment were carried out. Every session lasted about fifteen minutes. The initial distance between the expected optimal and the actual position of the generalized index of health of the patient was determined during each session. Then, the patient was asked to breath according to the metronome with a frequency of nineteen breaths per minute. After eight sessions the distance between the expected optimal and the actual position of the generalized index of health of the patient decreased by five times. Later on, during the ninth and tenth sessions the distance between the expected optimal and the actual positions of the generalized index of health of the patient stayed on one level. The general state of health of the patient improved and physical tiredness decreased.

### EXAMPLE 2

A male patient (L) was 63 years old and diagnosed with coronary heart disease, a painless form of late infrequent monotypic atrial extrasystoles, and atherosclerosis. He had general weakness and fatigability.

The patient was linked up with the apparatus for correction of functional state by four electrodes placed on his arms and his legs. The cycles of inhalation-exhalation per minute (i.e., frequency of breathing) were calculated imperceptibly for the patient during the recording of biopotentials. The frequency of cycles of inhalation-exhalation was nineteen times per minute. During the recording of electrocardiogram signal, the expected optimal and the actual position of the generalized index of health of the patient was observed on the monitor of the apparatus by the operator and the patient.

After switching on of the metronome with frequency of nineteen signals per minute, the patient was asked to breath in accordance with signals of the metronome. The generalized index of health deviated in the direction from the optimal position during the breathing according to the metronome. When the frequency of metronome and the coordinated breathing decreased to sixteen cycles per minute and then increased to twenty-two cycles per minute, the mutual position of the expected optimal and the actual generalized index of health was observed. The change in the path of movement of the index in the direction from an expected optimal index was noted in both cases.

The electrodes were transferred from the arms and the legs to the chest. The patient was told to make rhythmical exercises, namely circular motions with arms. At the same time, a position of the generalized index of health of the patient and its movement in the direction of optimal was observed on the monitor. After about three minutes, the patient finally chose a rhythm and range of motions of arms that caused the generalized index constantly move to the optimal index. It was suggested to make twelve sessions. Each session lasted about twenty minutes. In the next seven sessions, the result was steadied.

Ten sessions of treatment were carried out. The initial distance between the expected optimal and the actual position of the generalized index of health of the patient was determined in each session. Then, the patient was asked to breath in accordance with the metronome with a frequency of nineteen cycles per minute. After eight sessions the distance between the expected optimal and the actual position of the generalized index of health of the patient decreased by five times. Later on, during the ninth and tenth sessions the distance between the expected optimal and the actual position of the generalized index of health of the patient steadied on one level. The general state of health of the patient improved and physical tiredness decreased.

### EXAMPLE 3

A female patient (M) was a 22 year old athlete and diagnosed with an overstrain. The patient was linked up with the apparatus by four electrodes placed on her arms and legs. During the recording of an electrocardiogram signal, the expected optimal and the actual position of the generalized index of health of the patient was observed on the monitor of the apparatus.

A program of ten classical musical compositions of different composers each lasting sixty seconds was compiled and the patient was asked to listen to the program using a programmable audio player. The changes of position of the expected optimal and the actual position of the generalized index of health during the listening were evaluated to determine the results of the listening. The least distance between the expected optimal index and the actual position of the generalized index of health of the patient was during the listening of “O Sole Mio” performed by Pavarotti. The patient was told to listen to compositions performed by this singer, including “O Sole Mio,” every day for thirty minutes. After ten of the daily sessions under the conditions of the states of control study, the actual position of the generalized index of health of the patient corresponded with the expected optimal index.

### EXAMPLE 4

A male patient (D) was 43 years old and diagnosed with moderate arterial hypertension. Blood pressure was measured three times and made 165/102 mm of mercury. The patient was linked up with the apparatus by four electrodes placed on the chest. During the recording of an electrocardiogram signal, the expected optimal and the actual position of the generalized index of health of the patient was observed on the monitor of the apparatus.

The patient was asked to walk in place at a moderate pace. During the walking in place, changes of mutual dislocation of the expected optimal and the actual position of the generalized index of health of the patient were observed. After about seven minutes of walking in place, the distance between the indexes became less and became the least in about twenty minutes. After about twenty minutes, the distance between the indexes steadied on one level. The session was finished in thirty minutes. Then, blood pressure was again measured three times and made 142/94 mm of mercury. The patient was asked to take thirty minutes walks at a moderate pace every day. In ten sessions under these conditions of the states of control study, the essential approach of the actual position of the generalized index of health of the patient to the level of the expected optimal index was observed. After each session, the patient's blood pressure was measured three times and made 130/88 mm of mercury each time.

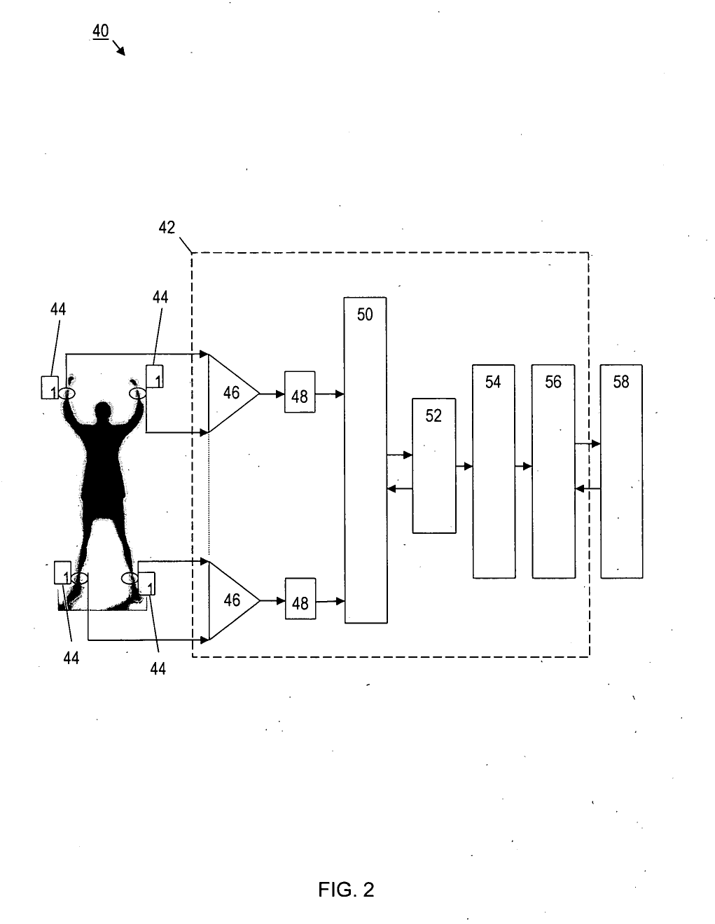

With reference to FIG. 2, an exemplary embodiment of the apparatus 40 for correction of a functional state of a person includes biosignal sensors 44, biosignal amplifiers 46, bandpass filters 48, an electronic switchboard 50, a microprocessor with a software support 52, an optical distributor device 54, an interface block 56, and a mini PC with a software support 58. The various components of the system of optimization are connected in series from the sensors to the mini PC, except the microprocessor 52 has feedback lines connected to the electronic switchboard 50.

The sensors 44 are linked to a patient to record the patient's biosignal. The type of biosignal recorded is determined by the type of sensor 44 and corresponding software support within the microprocessor 52. The amplifiers 46 provide intensification of the biosignal. The quantity of amplifiers 46 is determined by the quantity of sensors 44. There is one amplifier 46 for every two sensors 44. The intensified biosignal is supplied to the bandpass filters 48 in order to isolate the necessary part of a biosignal in the given frequency range. The bandpass filters 48 are switched by an electronic switchboard 50 which is operated by means of feedback from the microprocessor 52. The microprocessor 52 and its software support provide main functions of recording the biosignal, measurement of its parameters, automatic adjustment to the level of the biosignal, isolation of its necessary part, and numeralization with a given or adaptive frequency sampling. The optical distributor device 54 provides safety by isolating the patient from electricity. The interface block 56 provides interaction of the microprocessor 52 with the mini PC 58. This interaction is mainly aimed at transmission of a high quality numeralized biosignal to the mini PC 58.

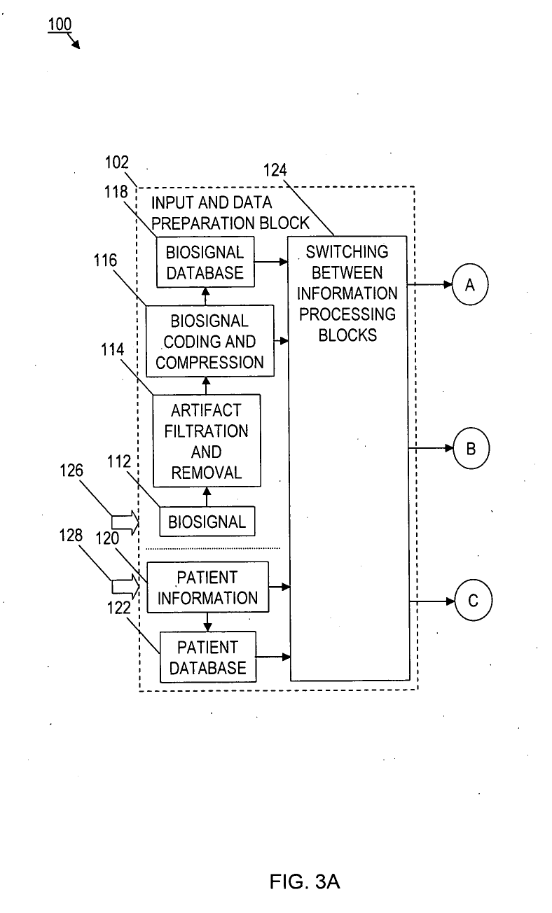

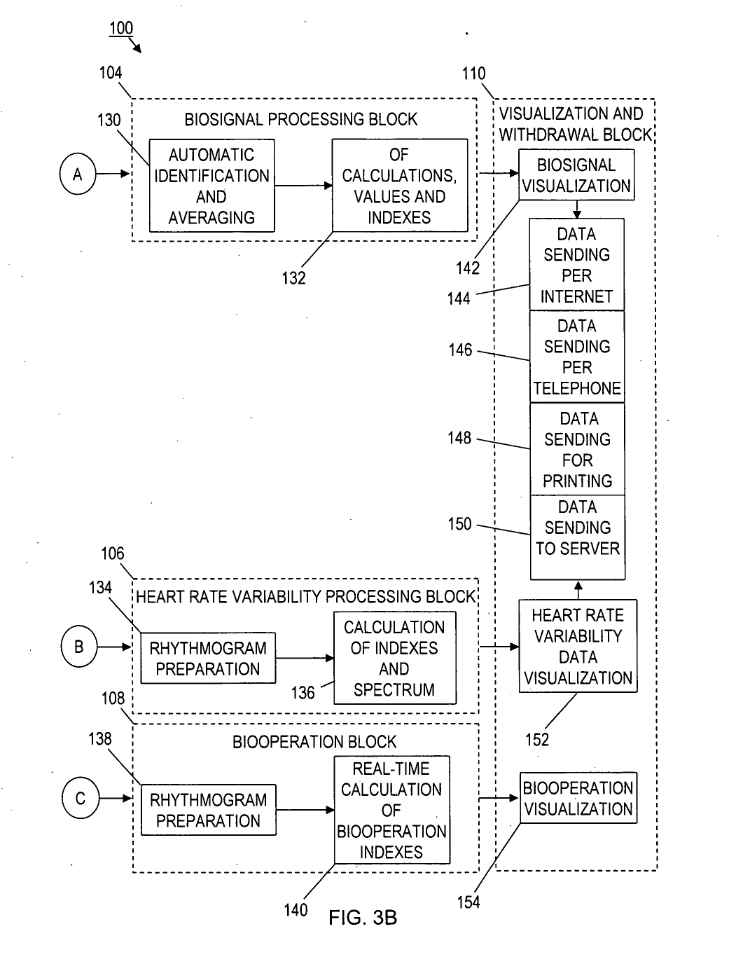

Individual information about a patient is registered and kept using the software support of mini PC 58. According to this registered individual information, an area of optimal state of the organism (i.e., patient) is determined. This area can be corrected by patient, taking into account, for example, experience of work with the system. The registered biosignal from the interface block 56 is exposed to prior software processing in the form of deleting of artifacts, filtration, and compression for further preservation and processing in the program (input and data preparation block 102, FIG. 3A).

With reference to FIGS. 3A and 3B, an exemplary embodiment of an operations and software support program 100 for the apparatus includes an input and data preparation block 102, a biosignal processing block 104, a heart rate variability block 106, a biooperation block 108, and a visualization and withdrawal block 110. The input and data preparation block 102 includes a biosignal block 112, an artifact filtration and removal block 114, a biosignal coding and compression block 116, a biosignal database 118, a patient information block 120, a patient database 122, a switching between information processing blocks block 124, a first input 126, and a second input 128. The biosignal processing block 104 includes an automatic identification and averaging block 130 and an of calculations, values, and indexes block 132. The heart rate variability block 106 includes a rhythmogram preparation block 134 and a calculation of indexes and spectrum block 136. The biooperation block 108 includes a rhythmogram preparation block 138 and an actual or real-time calculation of indexes and spectrum block 140. The visualization and withdrawal block 110 includes a biosignal visualization block 142, a data sending per Internet block 144, a data sending per telephone block 146, a data sending for printing block 148, a data sending to server block 150, a heart rate variability visualization 152, and a biooperation visualization block 154.

The biosignal 112 represents an electrocardiogram, pulsogram, phonocardiogram, etc. type biological signal. The artifact filtration and removal block 114 includes software and/or hardware components that filter and delete artifact in the biosignal 112. The biosignal coding and compression block 116 includes software and/or hardware components that code and compress the biosignal after it is free from artifacts. The biosignal database 118 includes software and/or hardware components that preserve the biosignal in a patient's database. The patient information block 120 includes software and/or hardware components that contain information about a patient (e.g., a patient's file). The patient database 122 includes software and/or hardware components that contain information about a patient in a database. The switching between information processing blocks block 124 includes software and/or hardware that switches between blocks of informational processing and biosignal visualizations. The first input 126 registers a biosignal (e.g., electrocardiogram, pulsogram, phonocardiogram, etc. signal) and transmits it to the computer. The second input 128 provides data about a patient to the computer (e.g., a patient's file).

The automatic identification and averaging block 130 includes software and/or hardware that makes an automatic identification of the points of control for biosignals. The an of calculations, values, and indexes block 132 includes software and/or hardware that makes calculations of biosignal parameters.

The rhythmogram preparation block 134 includes software and/or hardware that makes an automatic identification of rhythmogram received by processing the points of control of the biosignals The calculation of indexes and spectrum block 136 includes software and/or hardware that makes calculations and indices for spectrums according to the rhythmogram.

The rhythmogram preparation block 138 includes software and/or hardware that makes an automatic identification of the rhythmogram received by processing the points of control of the biosignals. The actual or real-time calculation of indexes and spectrum block 140 includes software and/or hardware that makes spectrum calculations in actual or real-time according to the spectrum's rhythmogram and generalized health index with its representation on the monitor.

The heart rate variability visualization 152 includes software and/or hardware that visualizes variability of heart rate on the monitor. The biooperation visualization block 154 includes software and/or hardware that visualizes generalized health index on the monitor in actual or real-time.

Processing of data in the program is provided according to a chosen regime and includes the following branches (i.e., processing blocks): 1) a biosignal processing block 104 that processes the biosignal for further visualization and calculation of its parameters and indexes, 2) a heart rate variability processing block 106 that processes the biosignal for further calculation of its variability (i.e., variability of biosignal (VBS)), formation of a spectrum, and determination of indexes of variability in the spectrum area; and 3) a biooperation processing block 108 that processes the biosignal in actual time for the calculation of the actual state of balance of regulator links in the organism of the patient.

The visualization and withdrawal block 110 provides a simple derivation of the biosignal on a display of the mini PC 58 (FIG. 2) for the purpose of quality control of the record and evaluation of parameters of the biosignal, biosignal visualization 142 of spectrum and spectral characteristics of the biosignal, visualization of actual and optimal functional state of a patient according to which the type and intensification of optimizing effect on the organism is selected. This block provides full range of informational processing of results of the work of the program including: sending data through the Internet 144, sending data through a telephone connection 146, sending data for printing 148, and sending data to a server 150 or another computer through channels of local connection (infrared, radio, cable).

The method and apparatus for its implementation provide effective correction of the functional state of a person. As described above, correction takes place taking into account an optimal state of an average person. This allows establishment of a sparing regime of correction for a person with health problems and/or reveals potential resources of a healthy person to correct a person's state trying to use these resources. Such an application of the exemplary technical solutions substantially enlarges the functional possibilities for the method and apparatus and allows its use, for example, in fitness or sports applications to predict and reach better results and other desirable effects.

The exemplary embodiments have been described with reference to the preferred embodiments. Obviously, modifications and alterations will occur to others upon reading and understanding the preceding detailed description. It is intended that the exemplary embodiments be construed as including all such modifications and alterations insofar as they come within the scope of the appended claims or the equivalents thereof.

1. A method of correction of a functional state of a subject organism, including:
a) measuring one or more biosignals reflecting a current functional state of the subject organism;
b) transforming and processing the one or more measured biosignals to provide a current generalized index of health of the subject organism;
c) evaluating a deviation of the current generalized index of health from a predetermined predicted optimal generalized index of health;
d) forming of external effect on the subject organism using a controllable factor of effect having one or more controllable parameters; and
e) regularly repeating the external effect until the deviation of the current generalized index of health from the predicted optimal generalized index of health is minimized and stabilized.

2. The method set forth in claim 1 wherein the controllable factor of effect includes at least one of an audible effect, a light effect, a temperature effect, a physical exercise, and a modulated breathing technique.

3. The method as set forth in claim 1 wherein the controllable factor of effect is chosen at least in part for ability to move the current generalized index of health toward the predicted optimal generalized index of health.

4. The method as set forth in claim 1 wherein a value for each of the one or more controllable parameters associated with the controllable factor of effect is chosen at least in part for ability to move the current generalized index of health toward the predicted optimal generalized index of health.

5. The method set forth in claim 1 wherein the one or more biosignals include at least one of a blood pressure of the subject organism, a cardiac output of the subject organism, a temperature of the subject organism, and a characteristic of a rennin-angiotensin system of the subject organism.

6. The method set forth in claim 1 wherein the subject organism is a patient.

7. The method set forth in claim 6 wherein the patient is a person.

8. The method set forth in claim 1, further including:
f) visualizing the deviation of the current generalized index of health from the predetermined predicted optimal generalized index of health on a screen of a monitor.

9. An apparatus for correction of a functional state of an organism, including:
a sensor adapted to measure a biosignal reflecting a current functional state of the organism;
an amplifier in communication with the sensor to intensify the biosignal;
a bandpass filter in communication with the biosignal amplifier to isolate a portion of the biosignal associated with a predetermined frequency range;
an electronic switchboard in communication with the bandpass filter for switching the biosignal;
a microprocessor in communication with the electronic switchboard to receive the switched biosignal from the electronic switchboard and to provide feedback to control operation of the electronic switchboard;
an optical distributor in communication with the microprocessor;
an interface block in communication with the optical distributor;
a computer in communication with the interface block; and
internal software located within at least one of the microprocessor and the computer that provides measuring, adaptation, and automatic control of one or more parameters of the biosignal.

10. The apparatus set forth in claim 9 wherein the internal software provides further processing of the biosignal for visualization on a screen of a monitor associated with the computer.

11. The apparatus set forth in claim 9 wherein the internal software provides further processing of the biosignal for calculation of parameters and indexes in order to calculate variability, formation of a spectrum area, and determination of indexes of variability in the spectrum area.

12. The apparatus set forth in claim 9 wherein the internal software provides further processing of the biosignal in actual time in order to calculate an actual state of balance of regulatory links in the organism and bioadaptive regulation of the functional state of the organism.

13. A method of correction of a physiological state of a subject organism, including:
a) providing a predicted optimal generalized index of health for the subject organism;
b) recording a biosignal reflecting a current physiological state of the subject organism;
c) transforming and processing the measured biosignal to determine a current generalized index of health of the subject organism;
d) evaluating a difference between the current generalized index of health and the predicted optimal generalized index of health; and
e) forming of external influence on the subject organism while observing the current generalized index of health by choosing a controllable factor of effect and values for controllable parameters associated with the corresponding controllable factor that change the current generalized index of health in a direction of the predicted optimal generalized index of health.

14. The method set forth in claim 13, further including:
f) visualizing the difference between the current generalized index of health and the predicted optimal generalized index of health on a screen of a monitor.

15. The method set forth in claim 13, further including:
f) periodically repeating the external influence using the chosen controllable factor of effect and chosen values for controllable parameters associated with the chosen controllable factor until the difference between the current generalized index of health and the predicted optimal generalized index of health is minimized and stabilized.

16. The method set forth in claim 15 wherein the external influence is repeated five to twenty times.

17. The method set forth in claim 15 wherein the external influence is provided for five to fifteen minutes each time.

18. The method set forth in claim 15 wherein a quantity of times the external influence sessions is periodically repeated and a duration for each time is determined at least in part by taking into account the current generalized index of health and goals of correction of the physiological state of the organism.

19. The method set forth in claim 13 wherein the controllable factor of effect includes at least one of an audible effect, a light effect, a temperature effect, a physical exercise, and a modulated breathing technique.

20. The method set forth in the subject organism is a person.
